(19) United States
(12) Patent Application Publication
(10) Pub. No.: US 2026/0157831 A1
(43) Pub. Date: Jun. 11, 2026

(54) DENTAL IRRIGATING DEVICE WITH STERILIZATION FUNCTION

(71) Applicants: Bixdo (SH) Healthcare Technology Co., Ltd., Shanghai (CN); GuangDong Bixdo Health Technology Co., Ltd., Shunde District, Foshan City (CN)

(72) Inventor: Jianan WANG, Shanghai (CN)

(73) Assignees: Bixdo (SH) Healthcare Technology Co., Ltd., Shanghai (CN); GuangDong Bixdo Health Technology Co., Ltd., Shunde District, Foshan City (CN)

(21) Appl. No.: 18/717,424

(22) PCT Filed: Aug. 24, 2023
(86) PCT No.: PCT/CN2023/114742

§ 371 Date: Jun. 7, 2024

(30) Foreign Application Priority Data
Dec. 27, 2022 (CN) 202223501757.8

(51) Int. Cl. A61C 17/02; A61L 2/10; A61L 103/45

(52) U.S. Cl. CPC A61C 17/02; A61L 2/10; A61L 2103/45; A61L 2202/11

(57) ABSTRACT

A dental irrigating device with a sterilization function is provided. The dental irrigating device comprises a dental irrigator 1 and a base 2. The dental irrigator 1 comprises a water tank 11, and a bottom of the water tank 11 is provided with a first transparent plate 111. The base 2 comprises a sterilization lamp 3, the dental irrigator 1 is disposed on the base 2, and the sterilization lamp 3 is positioned to correspond to the first transparent plate 111 and to perform a light sterilization on an interior of the water tank 11 of the dental irrigator 1. The sterilization lamp 3 emits sterilizing light through the first transparent plate 111 into the interior of the water tank 11, and performs sterilization and bacteriostasis inside the water tank 11.

### FIELD OF THE INVENTION

The present disclosure relates to the field of dental irrigators, and in particular, to a dental irrigating device with a sterilization function.

### BACKGROUND OF THE INVENTION

In recent times, an increasing number of people have become more conscious of oral hygiene, leading to greater emphasis on maintaining a clean oral cavity. Dental irrigators, in particular, have gained popularity among users. However, it's essential to periodically clean the dental irrigator itself. Due to its intricate design, certain parts—such as the water tank and internal components—are challenging to clean thoroughly. Over time, accumulated residue in these hard-to-reach areas can compromise the effectiveness of the device. While ordinary users typically disinfect the nozzle by placing it in a UV sterilization box, the overall size of the dental irrigator necessitates a larger sterilization box which is cumbersome, and also prevents comprehensive disinfection. The present disclosure aims to address this issue.

### SUMMARY OF THE INVENTION

In view of the above-mentioned shortcomings, the present disclosure provides a dental irrigating device with a sterilization function.

The dental irrigating device comprises a dental irrigator and a base. The dental irrigator comprises a water tank, and a bottom of the water tank is provided with a first transparent plate. The base comprises a sterilization lamp, the dental irrigator is disposed on the base, and the sterilization lamp is positioned to correspond to the first transparent plate and to perform a light sterilization on an interior of the water tank of the dental irrigator.

Preferably, the first transparent plate is a first glass sheet, and the first glass sheet is embedded in the bottom of the water tank.

Preferably, the base comprises an upper cover and a lower cover, the sterilization lamp is disposed within the base, and the upper cover of the base is provided with a second transparent plate at a position corresponding to the sterilization lamp.

Preferably, the second transparent plate is a second glass sheet, and the second glass sheet is embedded in the upper cover.

Preferably, a wireless charging base is further disposed within the base, an induction coil assembly is disposed within the dental irrigator and configured to perform an electromagnetic induction charging on the wireless charging base, and the induction coil assembly is connected to a battery of the dental irrigator.

Preferably, the sterilization lamp is a UV sterilization lamp.

Preferably, a bottom of the base is provided with at least one foot pad.

Preferably, a side portion and a top portion of the water tank are non-transparent.

The above technical solutions, either individually or in combination, exhibit the following beneficial effects.

The dental irrigator's water tank features a transparent plate, allowing the sterilization lamp to shine through, effectively sterilizing the interior of the water tank.

The base comprises the wireless charging base for recharging the dental irrigator's battery. During charging, it can still sterilize the water tank without disassembly of the tank. This convenience makes the process straightforward and user-friendly.

### BRIEF DESCRIPTION OF DRAWINGS

FIG. 1 shows a schematic view of a dental irrigating device with a sterilization function according to the present disclosure.

FIG. 2 shows a cross-sectional view along A-A in FIG. 1.

FIG. 3 shows an enlarged view of B in FIG. 2.

FIG. 4 shows an exploded view of a base and its lower cover in the dental irrigating device according to the present disclosure.

### REFERENCE NUMERALS

| | |
|---|---|
| Dental irrigator | 1 |
| Water tank | 11 |
| Battery | 12 |
| First transparent plate | 111 |
| Base | 2 |
| Lower cover of the base | 21 |
| Upper cover of the base | 22 |
| Second transparent plate | 23 |
| Foot pad | 24 |
| Sterilization lamp | 3 |
| Integrated circuit board | 4 |
| Wireless charging base | 5 |
| Power supply line | 6 |

### DETAILED DESCRIPTION OF THE INVENTION

The exemplified embodiments described below serve as illustrative examples. Those skilled in the art may envision other evident variations. The fundamental principles outlined in the subsequent description can be applied to other embodiments, variations, improvements, equivalents, and other technical solutions that remain consistent with the spirit and scope of this disclosure.

It is essential for technical experts to recognize that terms such as “longitudinal”, “horizontal”, “up”, “down”, “front”, “back”, “left”, “right”, “vertical”, “horizontal”, “top”, “bottom”, “inside”, and “outside” indicate the orientation or position relationship based on those shown in the accompanying drawings. It is only for the convenience of describing and simplifying the description, and does not indicate or imply that the device or component mentioned must have a specific orientation, be constructed and operated in a specific orientation, and cannot be construed as restricting the present disclosure.

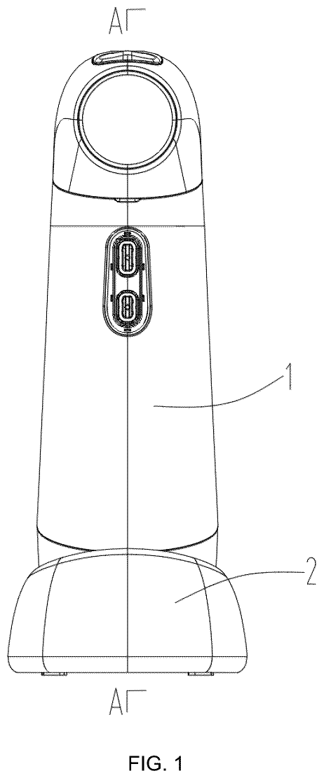

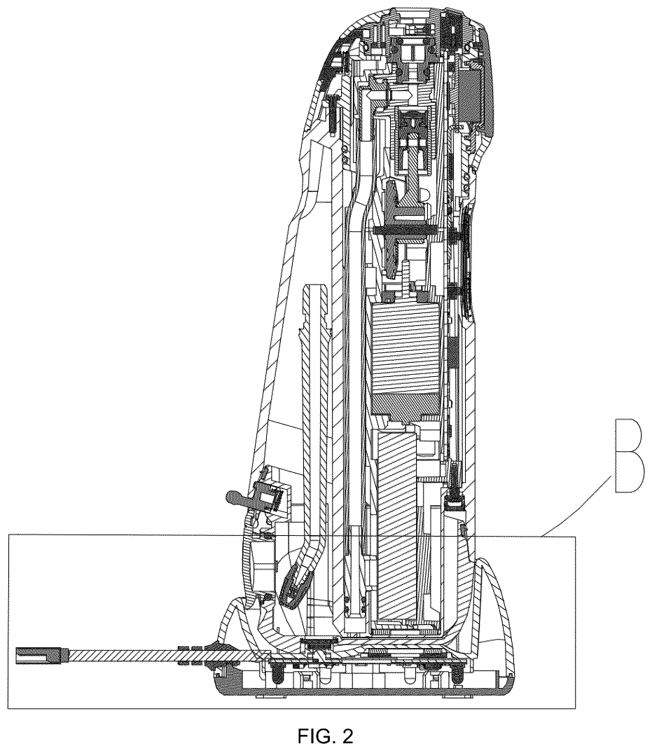

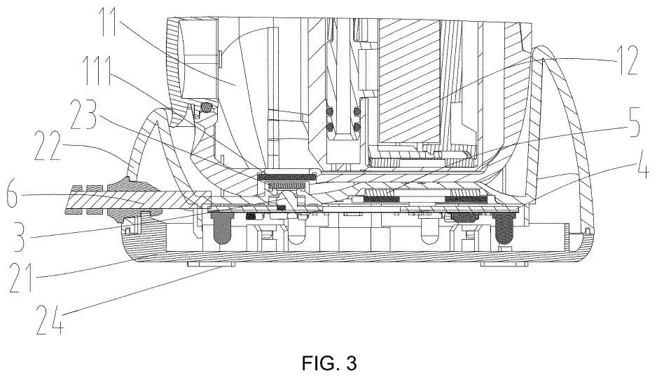

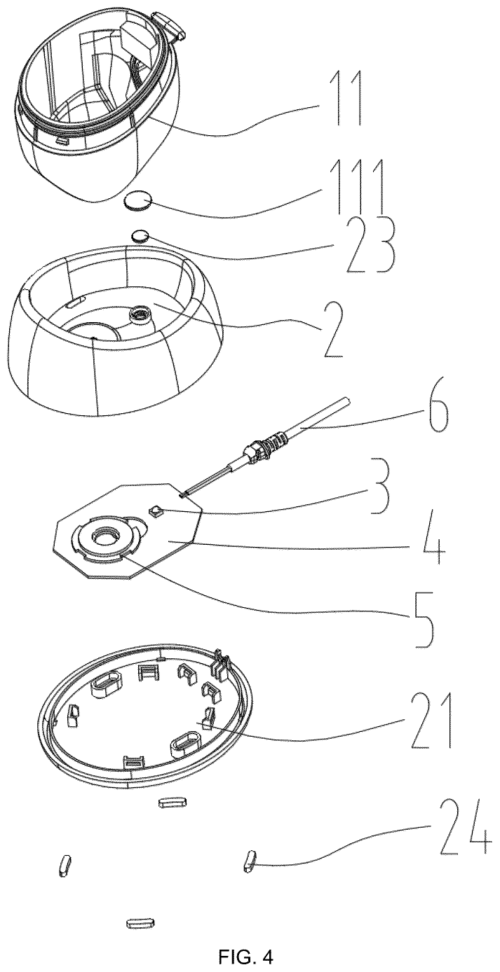

Referring to FIGS. 1-4, the present disclosure provides a dental irrigating device with a sterilization function. The dental irrigating device comprises a dental irrigator 1 and a base 2. The dental irrigator 1 comprises a water tank 11, and a bottom of the water tank 11 is provided with a first transparent plate 111. The base 2 comprises a sterilization lamp 3, the dental irrigator 1 is disposed on the base 2, and the sterilization lamp 3 is positioned to correspond to the first transparent plate 111 and to perform a light sterilization on an interior of the water tank 11 of the dental irrigator 1. The sterilization lamp 3 shoots sterilizing light through the first transparent plate 111 into the interior of the water tank 11 and performs sterilization and bacteriostasis inside the water tank 11.

Detailed description is provided below in conjunction with the accompanying drawings.

The sterilization lamp 3 may be a common ultraviolet (UV) sterilization lamp, and is configured to sterilize by ultraviolet light.

The bottom of the water tank 11 can be made of a transparent material as a whole or at least at the location of the sterilization lamp 3. That is, the water tank 11 can have either the entire bottom being the first transparent plate 111 or have only a portion of the bottom being the first transparent plate 111. In the latter configuration, the first transparent plate 111 is embedded in the bottom of the water tank 11. Preferably, the first transparent plate 111 is a first glass sheet, and the first glass sheet is embedded in the bottom of the water tank 11.

To enhance the waterproofing of the sterilization lamp 3, the sterilization lamp 3 is disposed within the base 2 or embedded in the base 2. The base 2 is provided with a second transparent plate 23 at a position corresponding to the sterilization lamp 3, and the second transparent plate 23 serves as a sealing plate for waterproof sealing.

Specifically, the base 2 comprises an upper cover 22 and a lower cover 21, the sterilization lamp 3 is disposed within the base 2, and the second transparent plate 23 is disposed at the upper cover 22 of the base 2. Further, the upper cover 22 is provided with a mounting hole, a mounting seat is disposed on the mounting hole, and the second transparent plate 23 is disposed at the mounting seat. In another embodiment, a hole is directly drilled in the upper cover 22, the second transparent plate 23 is embed within the drilled hole, and edges of the drilled hole are sealed.

Similar to the first transparent plate 111, the second transparent plate 23 is a second glass sheet and is embedded in the upper cover 22.

The dental irrigator 1 typically comes with a built-in rechargeable battery 12 (hereinafter, battery 12). After each charge, the battery 12 can be used multiple times. For the convenience of simultaneous charging during the sterilization and disinfection process, a wireless charging base 5 is further disposed within the base 2, an induction coil assembly is disposed within the dental irrigator 1 to perform an electromagnetic induction charging on the wireless charging base 5, and the induction coil assembly is connected to the battery 12 of the dental irrigator 1. When the dental irrigator 1 is disposed on the base 2, the sterilization lamp 3 utilizes the first transparent plate 111 and the second transparent plate 23 to irradiate and disinfect the water tank 11. The wireless charging base 5 wirelessly charges the battery 12.

In terms of electrical structures, the base 2 is provided with an integrated circuit board 4, and the wireless charging base 5 and the sterilization lamp 3 are both mounted on or connected to the integrated circuit board 4. The integrated circuit board 4 is powered by an external power supply line 6.

To enhance the stability of the base 2, a bottom of the base 2 is provided with at least one foot pad 24. Preferably, the foot pad 24 is a soft rubber pad.

In practical use, the ultraviolet light should ideally remain contained within the water tank 11 and not radiate outside the tank. Therefore, a side portion and a top portion of the water tank 11 should be non-transparent to prevent any discomfort for the user caused by any ultraviolet light exposure.

The above technical solutions, either individually or in combination, exhibit the following beneficial effects.

The bottom of the water tank 11 is provided with the first transparent plate 111, allowing the sterilization lamp 3 to shine through, effectively sterilizing the interior of the water tank 11.

The base 2 comprises the wireless charging base 5 for recharging the dental irrigator's battery 12. During charging, it can still sterilize the water tank 11 without disassembly of the tank. This convenience makes the process straightforward and user-friendly.

It should be understood by those skilled in the art that the embodiments of the present disclosure shown in the foregoing description are illustrative and do not limit the present disclosure. The purpose of the present disclosure has been fully and effectively accomplished. The functional and structural principles of the present disclosure have been shown and illustrated in the embodiments, which may be subject to any deformation or modification without departing from the stated principles.

1. A dental irrigating device with a sterilization function, comprising:
a dental irrigator, comprising a water tank, wherein a bottom of the water tank is provided with a first transparent plate; and
a base, comprising a sterilization lamp, wherein the dental irrigator is disposed on the base, and the sterilization lamp is positioned to correspond to the first transparent plate and to perform a light sterilization on an interior of the water tank of the dental irrigator.

2. The dental irrigating device according to claim 1, wherein the first transparent plate is a first glass sheet, and the first glass sheet is embedded in the bottom of the water tank.

3. The dental irrigating device according to claim 1, wherein the base comprises an upper cover and a lower cover, the sterilization lamp is disposed within the base, and the upper cover of the base is provided with a second transparent plate at a position corresponding to the sterilization lamp.

4. The dental irrigating device according to claim 3, wherein the second transparent plate is a second glass sheet, and the second glass sheet is embedded in the upper cover.

5. The dental irrigating device according to claim 1, wherein a wireless charging base is further disposed within the base, an induction coil assembly is disposed within the dental irrigator and configured to perform an electromagnetic induction charging on the wireless charging base, and the induction coil assembly is connected to a battery of the dental irrigator.

6. The dental irrigating device according to claim 1, wherein the sterilization lamp is a UV sterilization lamp.

7. The dental irrigating device according to claim 1, wherein a bottom of the base is provided with at least one foot pad.

8. The dental irrigating device according to claim 1, wherein a side portion and a top portion of the water tank are non-transparent.
